(19) United States
(12) Patent Application Publication
(10) Pub. No.: US 2012/0003207 A1
(43) Pub. Date: Jan. 5, 2012

(54) METHODS AND COMPOSITIONS FOR MODULATING PROLINE LEVELS

(75) Inventor: Mike A. Clark, Cheyenne, WY (US)

(73) Assignee: ONCOPHARMACOLOGICS, INC., Cheyenne, WY (US)

(21) Appl. No.: 13/235,098

(22) Filed: Sep. 16, 2011

Related U.S. Application Data
(63) Continuation of application No. 12/335,917, filed on Dec. 16, 2008, now abandoned.

(51) Int. Cl. A61K 38/43; A61P 35/00; A61K 31/7088; A61K 38/44; A61K 39/395

(52) U.S. Cl. 424/130.1; 424/94.1; 424/94.4; 424/94.3; 424/178.1; 514/44 A

(57) ABSTRACT

Methods and compositions for modulating amino acid levels in a subject are provided herein.

### TECHNICAL FIELD

The present disclosure is directed, inter alia, to methods for modulating amino acid levels in a subject and more particularly, to modulating proline levels for treating diseases such as cancer.

### BACKGROUND

Proline is not an essential component of the human diet, although it may be required for optimal growth (Jaksic et al., Am. J. Clin. Nutr. 52:307-312, 1990). Proline is an important component of skin collagen, thus proline requirements are elevated in severely burned patients (Jaksic et al., Am. J. Clin. Nutr., 54:408-413, 1991). Proline biosynthesis and oxidation is tightly regulated in mammalian cells. The initial step in proline catabolism is catalyzed by proline oxidases (also known as proline dehydrogenases), which convert proline to Δ¹-pyrroline-5-carboxylic acid (P5C) (reviewed in Adams and Frank, Ann. Rev. Biochem., 49:1005-1061, 1980). P5C is oxidized to glutamate by P5C dehydrogenase. Genetic abnormalities in proline oxidase and P5C dehydrogenase are associated with hyperprolinemic disorders in mice and humans (Raux et al., Hum Mol. Genet., 16(1):83-91, 2006; Bender et al., Am. J. Hum. Genet., 76:409-420, 2005).

Some tumor cells require nonessential amino acids to grow in vitro. It has been reported that melanomas, hepatomas, sarcomas and leukemia require arginine for growth (Sugimura et al., Melanoma Res., 2:191-196, 1992; Takaku et al., Int. J. Cancer, 51:244-249, 1992; and Miyazaki et al, Cancer Res., 50:4522-4527, 1990). In some cases, this requirement is due to a deficiency in arginosuccinate synthase. Administration of arginine deaminase eliminates arginine from the blood and kills tumor cells that require arginine for growth (J. B. Jones, “The Effect of Arginine Deiminase on Murine Leukemic Lymphoblasts,” Ph.D. Dissertation, The University of Oklahoma, pages 1-165, 1981). Similarly, deficiencies in asparagine synthetase in Acute Lymphoblastic Leukemias render these cancers susceptible to treatment with L-asparaginase (Park et al., Anticancer Res., 1:373-376, 1981).

### SUMMARY

The present disclosure provides agents that reduce proline levels in vivo and methods of using the agents to treat disorders such as cancers. Agents that reduce proline levels include, inter alia, enzymes that catabolize proline, compounds that increase the expression or activity of such enzymes, compounds that inhibit proline synthesis, and compounds that otherwise reduce levels of proline. The present disclosure also provides methods of treatment (e.g., methods of treating a cancer or a cancer symptom) by administering a proline reducing agent, and/or reducing dietary consumption of proline.

In some aspects, this disclosure features methods of treating a cancer or one or more cancer symptoms in a subject. The methods include administering to the subject an agent that reduces proline levels in the subject. The agent can be an enzyme such as proline hydroxylase. The enzyme can be modified to increase its circulating half life. For example, the enzyme can be modified to comprise an Fc region of an immunoglobulin, or a serum albumin. The enzyme can be linked to one or more polyethylene glycol (PEG) moieties (e.g., three or more PEG moieties). The one or more PEG moieties can have a molecular weight of about 5,000 to about 30,000 (e.g., a molecular weight of about 5,000, a molecular weight of 10,000, a or a molecular weight of about 20,000). The enzyme can be linked to one or more PEG moieties by a linking group selected from the group consisting of a succinimide group, an amide group, an imide group, a carbamate group, an ester group, an epoxy group, a carboxyl group, a hydroxyl group, a carbohydrate, a tyrosine group, a cysteine group, a histidine group and a combination thereof. The succinimide group can be succinimidyl succinate, succinimidyl propionate, succinimidyl carboxymethylate, succinimidyl succinamide, N-hydroxy succinimide or a combination thereof. The succinimide group can be succinimidyl succinate, succinimidyl propionate or a combination thereof.

The agent can be administered by a route selected from the group consisting of: orally, parenterally, intravenously, intramuscularly, subcutaneously, and intraperitoneally. The agent can be administered at or near a site of the cancer in the subject. The agent can be administered in a sustained release formulation. The subject can be a human. The cancer can be selected from the group consisting of an ovarian cancer, a colon cancer, a sarcoma, a lymphoma, a myeloma, a breast cancer, prostatic cancer, a skin cancer, an esophageal cancer, a liver cancer, a pancreatic cancer, a uterine cancer, a cervical cancer, a lung cancer, a bladder cancer, and a neural cancer. The agent can reduce levels of circulating proline by at least 10 μmol/L, 20 μmol/L, 40 μmol/L, 80 μmol/L, 100 μmol/L, or 120 mmol/L. The agent can be administered in an amount sufficient to reduce growth of cells of the cancer in the subject. The agent can be administered daily, weekly, every other week, or monthly.

In some aspects, this disclosure features methods of treating cancers or one or more cancer symptoms in a subject. The methods include reducing dietary proline consumption by the subject. The methods can further include administering a composition comprising an agent that reduces proline levels. The agent can be selected from the group consisting of an enzyme that reduces proline levels, a compound that increases the expression or activity of an enzyme that catabolizes proline, and an agent that inhibits proline synthesis. The agent can be proline hydroxylase.

This disclosure also features compositions for treating cancer or one or more cancer symptoms. The composition includes proline hydroxylase linked to one or more PEG moieties. The one or more PEG moieties can have a molecular weight of about 5,000 to about 30,000 (e.g., a molecular weight of about 5,000, a molecular weight of 10,000, a or a molecular weight of about 20,000). The enzyme can be linked to one or more PEG moieties by a linking group selected from the group consisting of a succinimide group, an amide group, an imide group, a carbamate group, an ester group, an epoxy group, a carboxyl group, a hydroxyl group, a carbohydrate, a tyrosine group, a cysteine group, a histidine group and a combination thereof. The succinimide group can be succinimidyl succinate, succinimidyl propionate, succinimidyl carboxymethylate, succinimidyl succinamide, N-hydroxy succinimide or a combination thereof. The succinimide group can be succinimidyl succinate, succinimidyl propionate or a combination thereof. The compositions further can include a second agent which is an anti-cancer agent selected from the group consisting of a chemotherapeutic drug and an antibody that induces cytotoxicity in the cancer.

In some aspects, this disclosure features kits for treating cancer or one or more cancer symptoms. The kits include a first agent that reduces proline levels, and a second agent, wherein the second agent is an anti-cancer agent selected from the group consisting of a chemotherapeutic drug and an antibody that induces cytotoxicity in the cancer. The first agent can be proline hydroxylase, proline oxidase, an antisense nucleic acid, or a proline analog.

The details of one or more embodiments of the invention are set forth in the accompanying drawings and the description below. Other features, objects, and advantages of the invention will be apparent from the description and drawings, and from the claims. All cited patents, and patent applications and references (including references to public sequence database entries) are incorporated by reference in their entireties for all purposes.

### DESCRIPTION OF THE DRAWINGS

FIG. 1 is a graph depicting percent survival of HT29 cells in the presence of prolinase (filled circles); boiled prolinase (open circles), and trypsin-treated prolinase (triangles).

FIG. 2 is a graph depicting percentages of viable cells (CACO2, filled circles; HT 29, open circles; COLO, filled triangles; 320 HSR, open triangles; and SK Mel 1 human melanoma, filled squares) in the presence of prolinase.

FIG. 3 is a graph depicting plasma proline concentrations in mice treated with prolinase.

FIG. 4 is a graph depicting tumor size in prolinase-treated mice (open circles) and control mice (filled circles).

Like reference symbols in the various drawings indicate like elements.

### DETAILED DESCRIPTION

The inventions described herein are based, in part, on the discovery that certain tumor cells require the amino acid proline for growth, i.e., the cells are proline auxotrophic. Treatment of proline-auxotrophic tumor cells with a proline depleting agent can induce cytotoxicity in the cells. Normal cells that retain the ability to synthesize proline are unaffected. Accordingly, the current disclosure provides proline reducing agents and methods of using the agents to induce cell cytotoxicity, e.g., therapeutically, to treat disease conditions such as cancers. Proline reducing agents include, without limitation, agents that degrade or catabolize proline (e.g., proline catabolic enzymes), agents that inhibit proline synthesis (e.g., inhibitors of proline synthetic enzymes), agents that increase the expression or activity of proline catabolic enzymes (e.g., nucleic acids encoding proline catabolic enzymes), or agents that otherwise produce lower levels of proline. Also provided are recombinant DNA molecules encoding the proline reducing agents, recombinant vectors and host cells including the DNA molecules, and therapeutic compositions including proline reducing agents. The therapeutic compositions can include biocompatible carriers or diluents. Proline reducing agents that are polypeptides (e.g., enzymes) can be modified to have an increased circulating half life in vivo and reduced immunogenicity. For example, an enzyme can be modified with a polypeptide that increases circulating half life, such as Fc, and/or modified with a biocompatible polymer such as polyethylene glycol.

### DEFINITIONS

Throughout the present disclosure, the following abbreviations may be used: PEG, polyethylene glycol; SS, succinimidyl succinate; SSA, succinimidyl succinamide; SPA, succinimidyl propionate; and NHS, N-hydroxy-succinimide.

“Polyethylene glycol” or “PEG” refers to mixtures of condensation polymers of ethylene oxide and water, in a branched or straight chain, represented by the general formula H(OCH2CH2)₂OH, wherein n is at least 4. “Polyethylene glycol” or “PEG” is used in combination with a numeric suffix to indicate the approximate weight average molecular weight thereof. For example, PEG-5,000 (PEGS) refers to polyethylene glycol molecules having an average molecular weight of about 5,000; PEG-12,000 (PEG12) refers to polyethylene glycol molecules having an average molecular weight of about 12,000; and PEG-20,000 (PEG20) refers to polyethylene glycol molecules having an average molecular weight of about 20,000.

As used herein, the terms “individual” and “subject” refer to an animal, in some embodiments a mammal, and in some embodiments a human.

As used herein, “modulation” means either an increase (stimulation) or a decrease (inhibition) in the expression or activity of a gene or gene product.

As used herein, the term “inhibit” refers to a reduction or decrease in a quality or quantity, compared to a baseline. For example, in the context of the present invention, inhibition of cell proliferation refers to a decrease in cell proliferation as compared to baseline. In some embodiments there is a reduction of about 30%, about 50%, about 75%, about 80%, about 85%, about 90%, about 95%, about 99%, and about 100%. Those of ordinary skill in the art can readily determine whether or not cell proliferation has been inhibited and to what extent.

As used herein, the term “biocompatible” refers to materials or compounds which are generally not injurious to biological functions and which will not result in any degree of unacceptable toxicity, including allergenic and disease states.

“Circulating half life” refers to the period of time, after injection of a composition (e.g., an enzyme that catabolizes proline or an enzyme that hydroxylates proline) into a patient, until a quantity of the composition has been cleared to levels one half of the original peak serum level. Circulating half-life may be determined in any relevant species, including humans or mice.

As used herein, the terms “covalently bonded”, “bonded” and “coupled” are used interchangeably and refer to a covalent bond linking a polypeptide to the PEG molecule, either directly or through a linker.

As used herein, the term “therapeutically effective amount” refers to an amount of a compound of the present invention effective to yield the desired therapeutic response. The therapeutically effective amount can vary with such factors as the particular condition being treated, the physical condition of the patient, the type of mammal or animal being treated, the duration of the treatment, the nature of concurrent therapy (if any), the specific formulations employed, and the structure of the compounds or its derivatives. In the context of treating a cancer, the term “therapeutically effective amount” refers to an amount of a composition that reduces the growth rate of cells of a cancer, or causes stasis or regression of a cancer, or is cytotoxic to cancer cells of a subject.

As used herein, the term “an amount effective to reduce circulating proline levels” refers to an amount of a compound administered to an individual that results in a reduced level of proline that is detectable. To determine an amount effective to reduce circulating proline levels, the individual's proline levels can be determined prior to treatment with an agent described herein, and then subsequent to treatment. The level of proline (e.g., in plasma or urine) can be quantified by routine methodologies including, for example, automated ion-exchange chromatography (see, e.g., Lepage et al., Clin. Chem. 43(12):2397-2402, 1997).

As used herein, the term “prophylactically effective amount” refers to an amount of an agent effective to yield the desired prophylactic response. The specific prophylactically effective amount can vary with such factors as the physical condition of the subject, the type of subject being treated, the duration of the treatment, the nature of concurrent therapy (if any), and the specific formulations employed and the structure of the agent.

As used herein “combination therapy” means that the individual in need of treatment is given another drug for the disease (e.g., cancer) in conjunction with an agent that reduces proline levels. Combination therapy can be sequential therapy where the individual is treated first with one or more drugs and then the other, or where the individual is given two or more drugs simultaneously.

As used herein, the phrases “proline deprivation” and “proline reduction” refer to a treatment regimen that involves the use of an agent that reduces, minimizes, or abolishes proline levels in the patient. In some embodiments, proline deprivation therapy is performed using an enzyme that catabolizes proline, as described in detail herein.

As used herein, the term “sample” refers to biological material from a patient. The sample assayed by methods described herein is not limited to any particular type. Samples include, as non-limiting examples, single cells, multiple cells, tissues, tumors, biological fluids, biological molecules, or supernatants or extracts of any of the foregoing. Examples include tissue removed for biopsy, tissue removed during resection, blood, urine, lymph tissue, lymph fluid, cerebrospinal fluid, mucous, and stool samples. The sample used will vary based on the assay format, the detection method and the nature of the tumors, tissues, cells or extracts to be assayed. Methods for preparing samples are well known in the art and can be readily adapted in order to obtain a sample that is compatible with the method utilized.

### Proline Reducing Agents

Agents suitable for reducing proline levels in a subject include polypeptide agents, such as enzymes, that catabolize or degrade proline, or that otherwise produce lowered levels of the amino acid, e.g., by an indirect mechanism. In some embodiments, a praline analog can be used as a competitive inhibitor to interfere with proline uptake or result in feed back inhibition of the proline synthetic enzymes. Enzymes useful for reducing proline levels include proline oxidases (also referred to as proline dehydrogenases) and proline hydroxylases. Table 1 provides a list of enzymes that can be used to reduce proline levels in a subject. Prolyl 4-hydroxylase (proline hydroxylase, EC 1.14.11.2) is particularly useful. Prolyl 4-hydroxylase catalyzes the hydroxylation of proline in -Xaa-Pro-Gly-triplets in collagens and other proteins with collagen-like sequences. The vertebrate enzyme is an α₂β₂ tetramer in which the α-subunits contribute to most parts of the two catalytic sites. The β-subunit is identical to the enzyme protein disulfide-isomerase (PDI, EC 5.3.4.1) and has PDI activity even when present in the prolyl 4-hydroxylase tetramer. See, Annunen et al., J. Biol. Chem., 272(28):17342-17348, 1997. Other enzymes that can be useful are amino acid decarboxylases, amino acid deaminases, or proline specific peptidases (e.g., 5-oxo-L-prolinase, X-prolyl-dipeptidyl aminopeptidase, proline iminopeptidase, prolidase (imidodipeptidase).

Proline oxidases catalyze the conversion of proline to pyrroline-5-carboxylate, or P5C. P5C is then converted to glutamate by P5C dehydrogenases. Deficiencies in proline oxidase activity and P5C dehydrogenase activity lead to hyperprolinemia in mice and humans and the failure of proline to support growth in bacteria (Adams and Frank, Ann. Rev. Biochem., 49:1005-1061, 1980).

TABLE 1
Exemplary Proline Reducing Enzymes

| | | Amino acid | Nucleotide | |
|---|---|---|---|---|
| | | sequence | sequence | |
| | | GenBank Acc. | GenBank Acc. | |
| Name | Organism | No. | No. | Comment |
| proline dehydrogenase | Homo sapiens | NP_057419 | NM_016335 | |
| (oxidase) 1 (PRODH) | | | | |
| proline dehydrogenase | Pan troglodytes | XP_525525 | XM_525525 | |
| (oxidase) 1 (PRODH) | | | | |
| proline dehydrogenase | Canis lupus | XP_534757 | XM_534757 | |
| (oxidase) 1 | familiaris | | | |
| proline dehydrogenase | Mus musculus | NP_035302 | NM_011172 | |
| similar to Proline oxidase, | Rattus | XP_001058756.1 | XM_001058756.1 | |
| mitochondrial precursor | norvegicus | | | |
| (Proline dehydrogenase) | | | | |
| similar to MGC115247 | Danio rerio | XP_700477.2 | XM_695385.2 | |
| protein (also known as | | | | |
| LOC571764) | | | | |
| AT5G38710 proline oxidase, | Arabidopsis | NP_198687.1 | NM_123232.2 | |
| putative/osmotic stress- | thaliana | | | |
| responsive proline | | | | |
| dehydrogenase, putative | | | | |
| Os10g0550900 hypothetical | Oryza sativa | NP_001065321.1 | NM_001071853.1 | |
| protein | Japonica Group | | | |
| proline dehydrogenase | Homo sapiens | NP_067055 | NM_021232 | |
| (oxidase) 2 (PRODH2) | | | | |
| proline dehydrogenase | Pan troglodytes | XP_524461.2 | XM_524461.2 | |
| (oxidase) 2 (PRODH2) | | | | |
| proline dehydrogenase | Canis lupus | XP_541686.2 | XM_541686.2 | |
| (oxidase) 2 | familiaris | | | |
| (PRODH2) | | | | |
| proline dehydrogenase | Mus musculus | NP_062419.2 | NM_019546.5 | |
| (oxidase) 2 | | | | |
| (PRODH2) | | | | |
| proline dehydrogenase | Rattus | XP_341826.2 | XM_341825.3 | |
| (oxidase) 2 | norvegicus | | | |
| (PRODH2) | | | | |
| zgc: 92040 | Danio rerio | NP_001002391.1 | NM_001002391.1 | |
| proline dehydrogenase; | Arabidopsis | NP_189701.3 | NM_113981.5 | |
| ERD5, (Early Responsive to | thaliana | | | |
| Dehydration 5; proline | | | | |
| oxidase) | | | | |
| proline dehydrogenase (PutA) | E. coli | AAB59985 | U05212 | proline |
| | | | | dehydrogenase |
| | | | | and delta-1- |
| | | | | pyrroline-5- |
| | | | | carboxylate |
| | | | | dehydrogenase |
| proline dehydrogenase (PutA) | Sinorhizobium | CAA69727 | Y08500 | bifunctional |
| | meliloti | | | proline |
| | (Rhizobium | | | dehydrogenase/ |
| | meliloti) | | | pyrroline-5- |
| | | | | carboxylate |
| | | | | dehydrogenase |
| proline dehydrogenase (PutA) | Klebsiella | AAB95478 | AF038838 | |
| | aerogenes | | | |
| trifunctional transcriptional | Pseudomonas | NP_747050 | NC_002947 | |
| regulator/proline | putida | | (genome | |
| dehydrogenase/pyrroline-5- | | | sequence) | |
| carboxylate dehydrogenase | | | | |
| mitochondrial proline oxidase | S. cerevisiae | AAA16631 | M18107 | |
| (PUTI) | | | | |
| Delta-1-pyrroline-5- | Homo sapiens | P30038 | | |
| carboxylate dehydrogenase, | | | | |
| mitochondrial | | | | |
| precursor (P5C | | | | |
| dehydrogenase)(ALDH4A1) | | | | |
| Delta-1-pyrroline-5- | Saccharomyces | NP_011902 | | |
| carboxylate dehydrogenase | cerevisiae | | | |
| (Put2p) | | | | |
| delta-1-pyrroline-5- | Schizosaccharo- | NP_595958.1 | NM_001021867.1 | |
| carboxylate dehydrogenase | myces pombe | | | |
| delta 1-pyrroline-5- | Kluyveromyces | XP_452670.1 | XM_452670.1 | |
| carboxylate dehydrogenase | lactis | | | |
| delta-1-pyrroline-5- | Aegilops tauschii | AAZ91472 | DQ154922 | |
| carboxylate dehydrogenase | | | | |
| (P5CDH) | | | | |
| prolyl 4-hydroxylase alpha | Homo sapiens | AAB71339 | U90441 | Catalyzes the |
| (II) subunit | | | | hydroxylation of |
| | | | | proline in -Xaa- |
| | | | | Pro-Gly- triplets |
| | | | | in collagen and |
| | | | | other proteins |
| | | | | with collagen- |
| | | | | like sequences. |
| | | | | Annunen et al., J. |
| | | | | Biol. Chem., |
| | | | | 272(28): 17342- |
| | | | | 17348, 1997) |
| prolyl 4-hydroxylase alpha (I) | Homo sapiens | NP_000908 | NM_000917 | Catalyzes the |
| subunit | | | | hydroxylation of |
| | | | | proline in -Xaa- |
| | | | | Pro-Gly- triplets |
| | | | | in collagen and |
| | | | | other proteins |
| | | | | with collagen- |
| | | | | like sequences. |
| | | | | Annunen et al., J. |
| | | | | Biol. Chem., |
| | | | | 272(28): 17342- |
| | | | | 17348, 1997 |
| prolyl hydroxylase domain- | Homo sapiens | NP_444274.1 | NM_053046.2 | Hyroxylates two |
| containing protein-1 (HIF | | | | proline residues |
| prolyl hydroxylase 1; PHD1; | | | | in a conserved |
| EGL9, C. Elegans, Homolog | | | | LxxLAP |
| of, 2; EGLN2) | | | | sequence motif |
| | | | | (Berra et al., |
| | | | | EMBO Rep., |
| | | | | 7(1): 41-45, |
| | | | | 2006) |
| prolyl hydroxylase domain- | Homo sapiens | NP_071334.1 | NM_022051.1 | Hyroxylates two |
| containing protein-2 (PHD2; | | | | proline residues |
| egl nine homolog 1 ; HIF | | | | in a conserved |
| prolyl hydroxylase 2) | | | | LxxLAP |
| | | | | sequence motif |
| | | | | (Berra et al., |
| | | | | EMBO Rep., |
| | | | | 7(1): 41-45, |
| | | | | 2006) |
| prolyl hydroxylase domain- | Homo sapiens | NP_071356.1 | NM_022073.3 | Hyroxylates two |
| containing protein-3 (PHD3; | | | | proline residues |
| egl nine homolog 3; HIF | | | | in a conserved |
| prolyl hydroxylase 3) | | | | LxxLAP |
| | | | | sequence motif |
| | | | | (Berra et al., |
| | | | | EMBO Rep., |
| | | | | 7(1): 41-45, |
| | | | | 2006) |

Proline reducing agents can be derived from a source that is of the same species as the subject to be treated, or from a heterologous species. For example, in some embodiments, a human subject is treated with a human enzyme (e.g., a human proline oxidase). In some embodiments, a human subject is treated with a non-human enzyme (e.g., a proline oxidase from a xenogeneic mammalian species, a proline oxidase from a plant species, or a proline oxidase from a bacterial species). In some cases, heterologous enzymes have beneficial properties that render them particularly suitable for therapeutic applications, such as the ability to be produced in large quantities, stability, high activity at physiological pH, lack of a requirement for co-factors not found in plasma, low Kₘ, and high Vₘₐₓ. In some embodiments, the agents exhibit long circulating half life and reduced antigenicity. Methods for modifying polypeptides to reduce their antigenicity and increase circulating half life in vivo are disclosed herein.

Useful polypeptide agents include, without limitation, the polypeptides disclosed in Table 1, as well as orthologs of these polypeptides from other species. Fragments or variants of the polypeptides that retain proline reducing activity are also useful. For example, variants may include one or more changes in the naturally occurring amino acid sequence, e.g., one or more changes in amino acid residues which are not essential for activity. Such variants are typically at least 80%, 85%, 90%, 95%, 98%, or 99% identical to the native sequence. The percent identity between two amino acid sequences can be determined as follows. First, the amino acid sequences are aligned using the BLAST 2 Sequences (Bl2seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq-i c:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical amino acid residue is presented in both sequences. The percent identity is determined by dividing the number of matches by the length of the amino acid sequence of a polypeptide of Table 1 followed by multiplying the resulting value by 100.

It is noted that the percent identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

In some embodiments, variant polypeptides have 5 or fewer or 3 or fewer substitutions (e.g., conservative substitutions), deletions, or insertions.

As discussed herein, the polypeptides may be conjugated to PEG, e.g., to increase their circulating half life and reduce antigenicity. The attachment of PEG to lysine residues in an enzyme may, in some cases, inactivate the enzyme. Thus, amino acid substitutions can be engineered at lysine residues to produce a protein that loses less of its enzymatic activity upon pegylation. Accordingly, the variant polypeptides described herein include polypeptides having certain amino acid substitutions in the polypeptide chain. These amino acid substitutions provide for a modified polypeptide that loses less activity upon pegylation; i.e., the reduction of enzyme activity following pegylation in the modified enzyme is less than the reduction of enzyme activity following pegylation in the unmodified enzyme. By eliminating pegylation sites at or adjacent to the catalytic region of an enzyme, optimal pegylation can be achieved while minimizing loss of activity. In some embodiments, lysine is substituted with glutamic acid, valine, aspartic acid, alanine, isoleucine, leucine or a combination thereof.

The invention also provides chimeric or fusion forms of the polypeptide agents. Chimeric polypeptide agents include, for example, a proline reducing enzyme linked to a heterologous polypeptide. In some embodiments, the heterologous polypeptide is a polypeptide that increases the circulating half-life of the chimeric polypeptide in vivo. The polypeptide that increases the circulating half-life may be a serum albumin, such as human serum albumin, or the Fc region of the IgG subclass of antibodies that lacks the IgG heavy chain variable region.

The polypeptide agents can be incorporated into pharmaceutical compositions and administered to a subject in vivo.

In some aspects, the invention also features variants of a polypeptide, e.g., which function as an agonist (mimetic) or as an antagonist. Agonists of proline catabolic enzymes are useful for reducing proline levels in a subject. Antagonists of proline synthetic enzymes can also be useful for reducing proline levels. Variants can be generated by mutagenesis, e.g., by introducing one or more discrete point mutations, inserting or deleting sequences or truncating a polypeptide. An agonist of a proline catabolic enzyme can retain substantially the same, or a subset, of the biological activities (e.g., proline oxidase activity) of the naturally occurring form of the enzyme.

Variants of proline catabolic or synthetic enzymes can be identified by screening combinatorial libraries of mutants, e.g., truncation mutants, for agonist or antagonist activity. Libraries of fragments e.g., N terminal, C terminal, or internal fragments, of a coding sequence of a proline related enzyme can be used to generate a variegated population of fragments for screening and subsequent selection of variants of the enzyme.

Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of proline related enzymes. Recursive ensemble mutagenesis (REM), a technique which enhances the frequency of functional mutants in the libraries, can be used in combination with the screening assays to identify polypeptide variants (Arkin and Yourvan, Proc. Natl. Acad. Sci. USA 89:7811-7815, 1992; Delgrave et al., Protein Engineering 6:327-331, 1993).

Cell based assays can be exploited to analyze a variegated polypeptide library. For example, a library of expression vectors can be transfected into a cell line, e.g., a proline auxotrophic cell line. The viability of the transfected cells in the presence of proline can be determined, to evaluate the proline reducing activity of the encoded library members. Plasmid DNA can then be recovered from the cells which exhibited reduced viability, and the individual clones further characterized.

In some aspects, the invention features methods of making a proline catabolic polypeptide, e.g., a peptide having a non-wild type activity, including an agonist or super agonist of a naturally occurring proline catabolic enzyme. The methods can include altering the sequence of a proline catabolic enzyme, for example, by substituting or deleting one or more residues of a non-conserved region, a domain, or residue disclosed herein, and testing the altered polypeptide for the desired activity.

The invention also features methods of making an antagonist of a proline synthetic enzyme. The methods include altering the sequence of a proline synthetic enzyme by substituting or deleting one or more residues of a non-conserved region, a domain, or residue disclosed herein, and testing the altered polypeptide for the desired activity.

In some aspects, the invention features methods of making a fragment or analog of a proline catabolic enzyme or a proline synthetic enzyme. The methods include altering the sequence, e.g., by substituting or deleting one or more residues of a proline catabolic enzyme or a proline synthetic enzyme and testing the altered polypeptide for the desired activity. For example, the sequence of a non-conserved region, or a domain or residue described herein can be altered, and the resulting polypeptide tested for the desired activity.

Genes encoding the enzymes described herein may be derived, cloned or produced from any source, including, for example, microorganisms or mammalian cells. A gene may be cloned from a mammalian source, including a human source, or from a microorganism.

Enzymes from heterologous sources (e.g., microorganisms) can be antigenic. Administered enzymes also may be rapidly cleared from the circulation. Antigenicity and short circulating half-life may be ameliorated by covalently modifying the enzyme with polyethylene glycol (PEG). An enzyme covalently modified with PEG (with or without a linking group) may be hereinafter referred to as “pegylated.” When compared to a native form of the enzyme, the pegylated form retains most of its enzymatic activity, is far less antigenic, has a greatly extended circulating half-life, and is more efficacious, e.g., in reducing proline levels, and in the treatment of cancers.

The invention also provides isolated or purified nucleic acid molecules that encode the proline reducing polypeptides described herein. An isolated nucleic acid molecule can include a nucleotide sequence which is at least 80%, 85%, 90%, 93%, 94%, 95%, 96%, 97%, 98%, 99% identical to a nucleotide sequence encoding a native proline reducing polypeptide (e.g., a proline reducing polypeptide disclosed in Table 1) or a portion of a native proline reducing polypeptide.

A nucleic acid molecule can include a sequence corresponding to a biologically active domain, region, or functional site described herein (e.g., a domain that mediates the proline reducing activity, such as proline oxidation). A nucleic acid molecule encoding a biologically active portion of a proline reducing polypeptide can be prepared by isolating the desired fragment of the nucleic acid encoding the biologically active portion of the polypeptide having proline reducing activity, expressing the biologically active portion of the polypeptide (e.g., by recombinant expression in vitro) and assessing the activity of the encoded portion. For example, a biologically active portion of proline oxidase can include a proline oxidase (dehydrogenase) domain.

The invention further encompasses nucleic acid molecules that differ from the wild type nucleotide sequence of proline reducing polypeptides described herein. Such differences can be due to degeneracy of the genetic code (and result in a nucleic acid which encodes the same polypeptide as those encoded by a nucleotide sequence disclosed herein). In some embodiments, an isolated nucleic acid molecule of the invention has a nucleotide sequence encoding a protein with an amino acid sequence which differs from a wild type sequence, by at least 1, but less than 5, 10, 20, 50, or 100 amino acid residues.

If alignment is needed for this comparison the sequences should be aligned for maximum homology. In some embodiments, the encoded proteins can differ from a wild type sequence by no more than 5, 4, 3, 2, or 1 amino acids.

Nucleic acids can be chosen for having codons that are preferred or non-preferred for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). Many orthologs, homologs, and allelic variants of the polypeptide and amino acid sequences described herein are known in the art. Additional orthologs, homologs, and variants can be identified using methods known in the art.

### Antisense Nucleic Acid Molecules, Ribozymes and Modified Nucleic Acid Molecules

In some aspects, the invention features isolated nucleic acid molecules which are antisense to a polypeptide involved in proline synthesis. For example, an antisense nucleic acid can target pyrroline 5-carboxylate (P5C) synthase. A nucleic acid sequence encoding human P5C synthase can be found in GenBank Accession No. U68758. An exemplary nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire coding strand of a target polypeptide, or to only a portion thereof. In some embodiments, the antisense nucleic acid molecule is antisense to a “noncoding region” of the coding strand of a nucleotide sequence encoding a polypeptide (e.g., the 5′ and 3′ untranslated regions). In some embodiments, the antisense oligonucleotide is complementary to the region surrounding the translation start site of the mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a protein (e.g., a protein that mediates proline synthesis) to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In some embodiments, the antisense nucleic acid molecule is an α-anomeric nucleic acid molecule. An α-anomeric nucleic acid molecule forms specific double-stranded hybrids with complementary RNA in which, contrary to the usual β-units, the strands run parallel to each other (Gaultier et al., Nucleic Acids. Res. 15:6625-6641, 1987). The antisense nucleic acid molecule can also comprise a 2′-o-methylribonucleotide (Inoue et al., Nucleic Acids Res. 15:6131-6148, 1987) or a chimeric RNA-DNA analogue (Inoue et al., FEBS Lett. 215:327-330, 1987).

In some embodiments, the antisense nucleic acid is a ribozyme. A ribozyme having specificity for a target nucleic acid can include a sequence having known catalytic sequence responsible for mRNA cleavage (see U.S. Pat. No. 5,093,246 or Haselhoff and Gerlach, Nature, 334:585-591, 1988). For example, a derivative of a Tetrahymena L-19 IVS RNA can be constructed in which the nucleotide sequence of the active site is complementary to the nucleotide sequence to be cleaved in a mRNA encoding a proline synthetic enzyme. See, e.g., Cech et al. U.S. Pat. No. 4,987,071; and Cech et al. U.S. Pat. No. 5,116,742. Alternatively, target mRNA can be used to select a catalytic RNA having a specific ribonuclease activity from a pool of RNA molecules. See, e.g., Bartel, D. and Szostak, J. W., Science, 261:1411-1418, 1993.

Gene expression can be inhibited by targeting nucleotide sequences complementary to the regulatory region of the gene of interest (e.g., a promoter and/or enhancers) to form triple helical structures that prevent transcription of the gene in target cells. See generally, Helene, Anticancer Drug Des. 6:569-84, 1991; Helene, Ann. N.Y. Acad. Sci., 660:27-36, 1992; and Maher, Bioassays, 14:807-15, 1992. The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

A nucleic acid molecule can be modified at the base moiety, sugar moiety or phosphate backbone to improve, e.g., the stability, hybridization, or solubility of the molecule. For non-limiting examples of synthetic oligonucleotides with modifications see Toulmé, Nature Biotech. 19:17, 2001, and Faria et al., Nature Biotech., 19:40-44, 2001. Such phosphoramidite oligonucleotides can be effective antisense agents.

For example, the deoxyribose phosphate backbone of the nucleic acid molecules can be modified to generate peptide nucleic acids (see Hyrup B. et al., Bioorganic & Medicinal Chemistry, 4: 5-23, 1996). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al., 1996, supra, and Perry-O'Keefe et al. Proc. Natl. Acad. Sci. 93: 14670-675, 1996.

PNAs can be used in therapeutic and diagnostic applications. For example, PNAs can be used as antisense or antigene agents for sequence-specific modulation of gene expression by, for example, inducing transcription or translation arrest or inhibiting replication. PNAs can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al., 1996, supra); or as probes or primers for DNA sequencing or hybridization (Hyrup B. et al., 1996, supra; Perry-O'Keefe, supra).

In some embodiments, the oligonucleotide may include other appended groups such as peptides (e.g., for targeting host cell receptors in vivo), or agents facilitating transport across the cell membrane (see, e.g., Letsinger et al., Proc. Natl. Acad. Sci. USA 86:6553-6556, 1989; Lemaitre et al., Proc. Natl. Acad. Sci. USA 84:648-652, 1987; PCT Publication No. WO88/09810) or the blood-brain barrier (see, e.g., PCT Publication No. WO89/10134). In addition, oligonucleotides can be modified with hybridization-triggered cleavage agents (see, e.g., Krol et al., Bio-Techniques, 6:958-976, 1988) or intercalating agents (see, e.g., Zon, Pharm. Res., 5:539-549, 1988). To this end, the oligonucleotide may be conjugated to another molecule, (e.g., a peptide, hybridization triggered cross-linking agent, transport agent, or hybridization-triggered cleavage agent).

Also provided herein are molecular beacon oligonucleotide primer and probe molecules having at least one region which is complementary to a target nucleic acid, two complementary regions one having a fluorophore and one a quencher such that the molecular beacon is useful for quantitating the presence of the nucleic acid in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

### RNAi

Double stranded nucleic acid molecules that can silence a gene (e.g., a gene encoding a polypeptide that mediates proline synthesis such as P5C synthase) also can be used as proline reducing agents. RNA interference (RNAi) is a mechanism of post-transcriptional gene silencing in which double-stranded RNA (dsRNA) corresponding to a gene (or coding region) of interest is introduced into a cell or an organism, resulting in degradation of the corresponding mRNA. The RNAi effect persists for multiple cell divisions before gene expression is regained. RNAi is therefore an extremely powerful method for making targeted knockouts or “knockdowns” at the RNA level. RNAi has proven successful in human cells, including human embryonic kidney and HeLa cells (see, e.g., Elbashir et al., Nature, May 24; 411(6836):494-8, 2001). In some embodiments, gene silencing can be induced in mammalian cells by enforcing endogenous expression of RNA hairpins (see Paddison et al., PNAS USA 99:1443-1448, 2002). In some embodiments, transfection of small (21-23 nt) dsRNA specifically inhibits gene expression (reviewed in Caplen, Trends in Biotechnology 20:49-51, 2002).

Briefly, RNAi is thought to work as follows. dsRNA corresponding to a portion of a gene to be silenced is introduced into a cell. The dsRNA is digested into 21-23 nucleotide siRNAs, or short interfering RNAs. The siRNA duplexes bind to a nuclease complex to form what is known as the RNA-induced silencing complex, or RISC. The RISC targets the homologous transcript by base pairing interactions between one of the siRNA strands and the endogenous mRNA. It then cleaves the mRNA ˜12 nucleotides from the 3′ terminus of the siRNA (reviewed in Sharp et al., Genes Dev., 15: 485-490, 2001; and Hammond et al., Nature Rev. Gen., 2: 110-119, 2001).

RNAi technology in gene silencing utilizes standard molecular biology methods. dsRNA corresponding to the sequence from a target gene to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Gene silencing effects similar to those of RNAi have been reported in mammalian cells with transfection of a mRNA-cDNA hybrid construct (Lin et al., Biochem Biophys Res Commun., 281(3):639-44, 2001), providing yet another strategy for gene silencing.

### Dietary Reduction of Proline

Proline reduction can also be achieved by reducing proline intake. Dietary proline deprivation can be prescribed for individuals diagnosed with, or at risk for, a cancerous disorder. In some embodiments, dietary proline reduction can be practiced in a subject who is also receiving treatment with a proline reducing agent described herein.

To reduce proline levels by dietary means, a subject takes a diet that is low in, or devoid of, proline. In some embodiments, this is achieved through a diet having defined amino acid mixtures that are devoid of proline, e.g., as described in Jaksic et al., Am. J. Clin Nutr. 52:307-312, 1990. Dietary deprivation has been shown to cause significant reductions in plasma proline concentrations in humans (see Jaksic et al., supra). In some embodiments, a subject takes a low proline (e.g., a proline-free or nearly proline-free) diet for at least 2 weeks, 4 weeks, 4 months, 8 months, or one year. In some embodiments, a cancer patient takes a low proline diet for a period of time sufficient to allow tumor stasis or regression.

### Polyethylene Glycol

An enzyme described herein (e.g., a proline hydroxylase) can be pegylated to increased the circulating half-life and/or reduce antigenicity. There are many PEGs available that differ in their molecular weight and linking group. These PEGs can have varying effects on the antigencity, immunogenicity and circulating half-life of a protein (Zalipsky, S, and Lee, C. Polyethylene Glycol Chemistry: Biotechnical and Biomedical Applications. Pp. 347 370, Plenum Press, New York, 1992; Monfardini, C., et. al., Bioconjugate Chem. 6:62-69, 1995; Delgado C; Francis G E; Fisher D. The uses and properties of PEG-linked proteins. Crit. Rev. Ther. Drug Carrier Sys., 9:249-304, 1992.)

In some embodiments, each polyethylene glycol molecule has an average molecular weight of 5,000, from about 5,000 to about 10,000, from about 10,000 to about 50,000; from about 12,000 to about 40,000, from about 15,000 to about 30,000; and about 20,000.

The PEG moiety may be a branched or straight chain. In some embodiments, the PEG is a straight chain. Increasing the molecular weight of the PEG generally tends to decrease the immunogenicity of the enzyme. The polyethylene glycols having the molecular weights described in the present invention may be used in conjunction with an enzyme, and, optionally, a biocompatible linking group, to treat neoplastic diseases.

### Pegylation

An enzyme may be covalently bonded to PEG via a biocompatible linking group, using methods known in the art, as described, for example, by Park et al, Anticancer Res., 1:373-376 (1981); and Zaplipsky and Lee, Polyethylene Glycol Chemistry: Biotechnical and Biomedical Applications, J. M. Harris, ed., Plenum Press, NY, Chapter 21 (1992), the disclosures of which are hereby incorporated by reference herein in their entirety.

The linking group used to covalently attach PEG to an enzyme may be any compatible linking group. In some embodiments the linking group is a biocompatible linking group. “Biocompatible” indicates that the compound or group is non-toxic and may be utilized in vitro or in vivo without causing injury, sickness, disease or death. PEG can be bonded to the linking group, for example, via an ether bond, an ester bond, a thiol bond or an amide bond. Suitable linking groups include, for example, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine. In some embodiments the linking group is an ester group and/or a succinimide group. In some embodiments, the linking group is SS, SPA, SCM, SSA or NHS.

The particular linking groups do not appear to influence the circulating half-life of a pegylated enzyme or its specific enzyme activity. However, if a linking group is used, in some embodiments it is important to use a biocompatible linking group. The PEG which is attached to the protein may be either a single chain, as with SS-PEG, SPA-PEG and SC-PEG, or a branched chain of PEG may be used, as with PEG2-NHS.

Alternatively, an enzyme may be coupled directly to PEG (i.e., without a linking group) through an amino group, a sulfhydryl group, a hydroxyl group or a carboxyl group. In some embodiments, PEG is coupled to lysine residues on an enzyme.

The attachment of PEG to an enzyme increases the circulating half-life of the enzyme. The number of PEG molecules on the enzyme appear to be related to the circulating half-life of the enzyme, while the amount of retained enzymatic activity appears related to the average molecular weight of the PEG used. Increasing the number of PEG units on an enzyme decreases the enzymatic activity of the enzyme. Also, it is known that some PEG formulations are difficult to produce and yield relatively low amounts of product. Thus, to achieve an efficacious product, in some embodiments, a balance needs to be achieved among circulating half-life, antigenicity, efficiency of production, and enzymatic activity.

Generally, PEG is attached to a primary amine of an enzyme. Selection of the attachment site of polyethylene glycol on the enzyme is determined by the role of each of the sites within the active domain of the protein, as would be known to the skilled artisan. From 1 to about 30 PEG molecules may be covalently bonded to an enzyme. In some embodiments, an enzyme is modified with about 3 to about 10, or 7 to about 15 PEG molecules, from about 9 to about 12 PEG molecules. In some embodiments, about 30% to about 70% of the primary amino groups in an enzyme are modified with PEG, about 40% to about 60%, about 45% to about 55%, and about 50% of the primary amino groups in an enzyme are modified with PEG. In some embodiments, when PEG is covalently bonded to the end terminus of an enzyme, only 1 PEG molecule is utilized. Increasing the number of PEG units on an enzyme increases its circulating half life. However, increasing the number of PEG units decreases the specific activity of the enzyme. Thus, in some embodiments a balance needs to be achieved between the two, as would be apparent to one skilled in the art in view of the present disclosure.

In some embodiments, the linking groups attach to a primary amine of an enzyme via a maleimide group. Once coupled with the enzyme, SS-PEG has an ester linkage next to the PEG, which may render this site sensitive to serum esterase, which may release PEG from the enzyme in the body. SPA-PEG and PEG2-NHS do not have an ester linkage, so they are not sensitive to serum esterase.

In some embodiments, the linking group is a linking group disclosed in U.S. Pat. No. 6,737,259, which is incorporated herein by reference in its entirety.

### Methods of Treatment

In some embodiments, the present invention provides methods of treating cancer or a cancer symptom, or treating an individual at risk for cancer, by reducing proline levels in the individual. The methods include administering to the individual a therapeutically or prophylactically effective amount of an agent that reduces proline levels, such as an agent that catabolizes proline (e.g., an enzyme that catabolizes proline).

The methods and compositions described herein are useful, for example, for reducing growth, causing cytotoxicity, or causing regression or stasis of neoplastic cells that are sensitive to proline levels. As described herein, a significant proportion of human colon carcinomas are auxotrophic for proline. Therefore, depriving such cells of proline reduces cell survival in vitro and in vivo. The proline reducing agents described herein are suitable for treating any cancerous disorder in which the cancer cells exhibit heightened sensitivity to reduced proline levels. In some instances, the sensitivity is caused by the absence of a proline synthetic enzyme, such as P5C reductase or P5C synthase. The methods of treatment described herein can include determining whether an individual's tumor includes cells that are auxotrophic for proline (e.g., by evaluating growth of the tumor cells in vitro, or by examining expression of a proline synthetic enzyme in the tumor cells to identify tumors that are deficient for proline synthesis), and deciding whether or not to administer a proline reducing agent. Methods also can include monitoring the subject's proline level and/or monitoring tumor size.

Polypeptide agents (e.g., enzymes such as proline hydroxylase) can be provided as compounds that include the polypeptide covalently bonded via a linking group to PEG, wherein each PEG molecule has an average molecular weight of from about 5,000 to about 30,000. In some embodiments the enzyme is modified with two or more polyethylene glycol molecules, each molecule having an average molecular weight of about 5,000 to about 30,000, e.g., about 20,000. In some embodiments the linking group is selected from the group consisting of a succinimide group, an amide group, an imide group, a carbamate group, an ester group, an epoxy group, a carboxyl group, a hydroxyl group, a carbohydrate, a tyrosine group, a cysteine group, a histidine group and combinations thereof. In some embodiments the linking group is succinimidyl succinate. In some embodiments from about 7 to about 15 polyethylene glycol molecules are linked to the enzyme. In some embodiments from about 9 to about 12 polyethylene glycol molecules are linked to the enzyme.

In some embodiments, the methods further can include administering a therapeutically effective amount of an additional anti-cancer agent prior to, simultaneously, or following administration of the proline reducing agent.

A therapeutically effective amount of one of the agents of the present invention is an amount that is effective to reduce proline levels in a subject. Generally, treatment is initiated with small dosages which can be increased by small increments until the optimum effect under the circumstances is achieved. Generally, a therapeutic dosage of an agent of the present invention may be from about 0.001 to about 200 mg/kg twice a week to about once every two weeks. For example, the dosage may be about 0.1 mg/kg once a week as a 2 ml intravenous injection to about 20 mg/kg once every 3 days. The compounds can be administered in one dose, continuously or intermittently throughout the course of treatment. The agent may be administered several times each day, once a day, once a week, or once every two weeks.

In some embodiments, a proline-reducing enzyme is administered in a weekly dose of at least about 40 IU/m², at least about 80 IU/m², at least about 160 IU/m², or at least about 200 IU/m². In some embodiments, a proline-reducing agent is administered in a weekly dose that lowers plasma proline levels by at least 5%, 10%, 20%, 30%, 40%, 50%, 60%, 70%, 80%, or 90%. In some embodiments, the dose administered lowers plasma levels of proline, which typically range from 90 to 150 μmol/L, by at least 10 μmol/L, 20 μmol/L, 40 μmol/L, 80 μmol/L, 100 μmol/L, or 120 μmol/L.

Methods of determining the most effective means and dosage of administration are well known to those of skill in the art. In some embodiments twice weekly dosing over a period of at least several weeks is used. Often, the proline reducing agent will be administered for extended periods of time and may be administered for the lifetime of the individual, e.g., in order to suppress tumor growth, prevent recurrence of a tumor, or to reduce a cancer symptom. Methods of determining the most effective means and dosage of administration are well known to those of skill in the art. Single or multiple administrations can be carried out with one dose level and pattern being selected by the administrator.

The dosage administered will, of course, vary depending upon known factors, such as the pharmacodynamic characteristics of the particular agent and its mode and route of administration; the age, health and/or weight of the individual; the nature and extent of the symptoms; the kind of concurrent treatment; the frequency of treatment; the symptoms exhibited by the individual, and the effect desired.

A proline reducing agent may be administered in admixture with suitable pharmaceutical diluents, extenders, excipients, or carriers (collectively referred to herein as a pharmaceutically acceptable carrier) selected with respect to the intended form of administration and as consistent with conventional pharmaceutical practices. For example, in some embodiments, a proline reducing agent which is a polypeptide agent (e.g., a proline catabolizing enzyme) is mixed with a phosphate buffered saline solution, or any other appropriate solution known to those skilled in the art, prior to injection. The polypeptide formulation may be administered as a solid (lyophilate) or as a liquid formulation, as desired.

The compositions of the present invention are formulated according to the mode of administration to be used. In cases where pharmaceutical compositions are injectable pharmaceutical compositions, they are sterile, pyrogen free and particulate free. In some embodiments the compositions are isotonic formulations. In some embodiments additives for isotonicity can include one or more of sodium chloride, dextrose, mannitol, sorbitol and lactose. In some embodiments, the compositions are provided as isotonic solutions such as phosphate buffered saline. Stabilizers for the compositions include gelatin and albumin in some embodiments.

The in vivo means of administration of the agents described herein will vary depending upon the intended application. As one skilled in the art will recognize, administration of a proline reducing agent can be carried out, for example, by inhalation or suppository or to mucosal tissue such as by lavage to vaginal, rectal, urethral, buccal and sublingual tissue, orally, topically, intranasally, intraperitoneally, parenterally, intravenously, intralymphatically, intratumorly, intramuscularly, interstitially, intra-arterially, subcutaneously, intraoccularly, intrasynovial, transepithelial, and transdermally. The agents also can be administered at or near a site of cancer in the subject. The agents can be administered in oral dosage forms as tablets, capsules, pills, powders, granules, elixirs, tinctures, suspensions, syrups, and emulsions. In some embodiments, the agent is administered in a sustained release formulation. The agents may also be administered in intravenous (bolus or infusion), intraperitoneal, subcutaneous, or intramuscular form, all using dosage forms well known to those of ordinary skill in the pharmaceutical arts.

The proline reducing agents described herein are useful for treating cancers (e.g., cancers in which the cells are auxotrophic for proline). Examples of cancers include, but are not limited to, colon cancer, breast cancer, skin cancer, bone cancer, prostate cancer, liver cancer, lung cancer (e.g., small cell lung cancer (SCLC) and non-small cell lung cancer (NSCLC) such as squamous (epidermoid) carcinoma, adenocarcinoma (including bronchoalveolar), and large-cell (undifferentiated) carcinoma), brain cancer, cancer of the larynx, gallbladder, pancreas, rectum, parathyroid, thyroid, adrenal, neural tissue, head and neck, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, islet cell tumor, primary-brain tumor, acute and chronic lymphocytic and granulocytic tumors, hairy-cell tumor, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, leiomyoma tumor, cervical dysplasia and in situ carcinoma, neuroblastoma, retinoblastoma, soft tissue sarcoma, malignant carcinoid, topical skin lesion, mycosis fungoides, rhabdomyosarcoma, Kaposi's sarcoma, osteogenic and other sarcoma, malignant hypercalcemia, renal cell tumor, polycythermia vera, adenocarcinoma, glioblastoma multiforma, medulloblastoma, leukemias (e.g., acute myeloid leukemia, acute promyelocytic leukemia, acute lymphoblastic leukemia, chronic myelogenous leukemia), lymphomas (e.g., Hodgkin's disease and non-Hodgkin's lymphomas), malignant melanomas, epidermoid carcinomas, and other carcinomas and sarcomas. Methods described herein are particularly useful for treating colon cancer.

### Combination Therapy

Proline deprivation therapy as described herein may additionally be combined with other anti-cancer compounds to provide a combination treatment regimen. Any known anti-cancer agent may be combined with a proline reducing agent, as long as the combination does not eliminate the proline reducing activity of the agent. In some cases, combination therapy may be more effective than therapy with either agent individually.

Combination therapy can be sequential (i.e., treatment with one agent first and then the second agent), or it can involve treatment with both agents at the same time. The sequential therapy can be within a reasonable time after the completion of the first therapy before beginning the second therapy. The treatment with both agents at the same time can be in the same daily dose or in separate doses. For example, in some embodiments, treatment with one agent occurs on day 1 and with the other on day 2. The exact regimen will depend on the cancer or cancer symptom being treated, the stage of disease, and the response to the treatment.

In some embodiments, proline reducing therapy is used in combination with an additional cancer therapy. Cancer therapies including dendritic cell therapy, chemokines, cytokines (i.e., cytokines such as TNF-beta or TNF-alpha), chemotherapeutic agents (e.g., adenosine analogs (e.g., cladribine, pentostatin), alkyl sulfanates (e.g., busulfan)), anti-tumoral antibiotics (e.g., bleomycin, dactinomycin, daunorubicin, doxorubicin, epirubicin, idarubicin, mitoxantrone, mitomycin), aziridines (e.g., thiotepa), camptothecin analogs (e.g., irinotecan, topotecan), cryptophycins (e.g., cryptophycin 52, cryptophicin 1), dolastatins (e.g., dolastatin 10, dolastatin 15), enedyine anticancer drugs (e.g., esperamicin, calicheamicin, dynemicin, neocarzinostatin, neocarzinostatin chromophore, kedarcidin, kedarcidin chromophore, C-1027 chromophore, and the like), epipodophyllotoxins (e.g., etoposide, teniposide), folate analogs (e.g., methotrexate), maytansinoids (e.g., maytansinol and maytansinol analogues), microtubule agents (e.g., docetaxel, paclitaxel, vinblastine, vincristine, vinorelbine), nitrogen mustards (e.g., chlorambucil, cyclophosphamide, estramustine, ifosfamide, mechlorethamine, melphalan), nitrosoureas (e.g., carmustine, lamustine, streptoxacin), nonclassic alkylators (e.g., altretamine, dacarbazine, procarbazine, temozolamide), platinum complexes (e.g., carboplatin, cisplatin), purine analogs (e.g., fludarabine, mercaptopurine, thioguanine), pyrimidine analogs (e.g., capecitabine, cytarabine, depocyt, floxuridine, fluorouracil, gemcitabine), substituted ureas (e.g., hydroxyurea); anti-angiogenic agents (e.g., canstatin, troponin I), biologic agents (e.g., ZD 1839, virulizin and interferon), antibodies and fragments thereof (e.g., anti EGFR, anti-HER-2/neu, anti-KDR, IMC-C225), anti-emetics (e.g., lorazepam, metroclopramide, and domperidone), epithelial growth factor inhibitors (e.g., transforming growth factor beta 1), anti-mucositic agents (e.g., dyclonine, lignocaine, azelastine, glutamine, corticoid steroids and allopurinol), anti-osteoclastic agents (e.g., bisphosphonates (e.g., etidronate, pamidronate, ibandronate, and osteoprotegerin)), hormone regulating agents (e.g., anti-androgens, LHRH agonists, anastrozole, tamoxifen), hematopoietic growth factors, anti-toxicity agents (e.g., amifostine), kinase inhibitors (gefitinib, imatinib), and mixtures of two or more thereof.

In some embodiments, a proline reducing agent described herein is administered to a subject in conjunction with a cancer treatment such as a surgical procedure, radiation therapy and/or ablation therapy (e.g., laser therapy, infrared therapy and the like).

Agents described herein can be combined with packaging material and sold as a kit for reducing proline levels in a subject. Components and methods for producing articles of manufactures are well known. The articles of manufacture may combine one or more agents described herein (e.g., proline hydroxylase or pegylated proline hydroxylase). In addition, the articles of manufacture may further include reagents for measuring proline levels, additional chemotherapy agents, and/or other useful reagents for reducing levels of protein or treating cancer or one or more cancer symptoms. Instructions describing how the various reagents can be used also may be included in such kits.

The invention will be further described in the following examples, which do not limit the scope of the invention described in the claims.

### EXAMPLES

### Example 1

### Identification of Proline Auxotrophic Human Cancer Cells

Auxotrophic human tumor cell lines were identified by culturing various lines in minimal essential media supplemented with dialyzed calf serum, and testing cells for growth following the addition of nonessential amino acids. Thirteen human colon carcinoma cell lines failed to grow in minimal essential medium unless proline was provided. These cell lines were the following: HT29, COLO, 320HSR, DLD1, HCT15, HCT116, LOVO, LS123, LS174T, LS180, NCIH548, SKCO1, and SW48. Six colon carcinoma biopsies were tested and also found to require proline for growth, indicating a high incidence of proline auxotrophy in colon carcinoma.

RT-PCR was performed on mRNA isolated from human colon cancer cell lines to identify the component responsible for the proline auxotrophy. Pyrrolin 5 carboxylate synthase mRNA was lacking in all of the colon cancer cells tested.

### Example 2

### Prolinase Inhibition of Human Cancer Cells

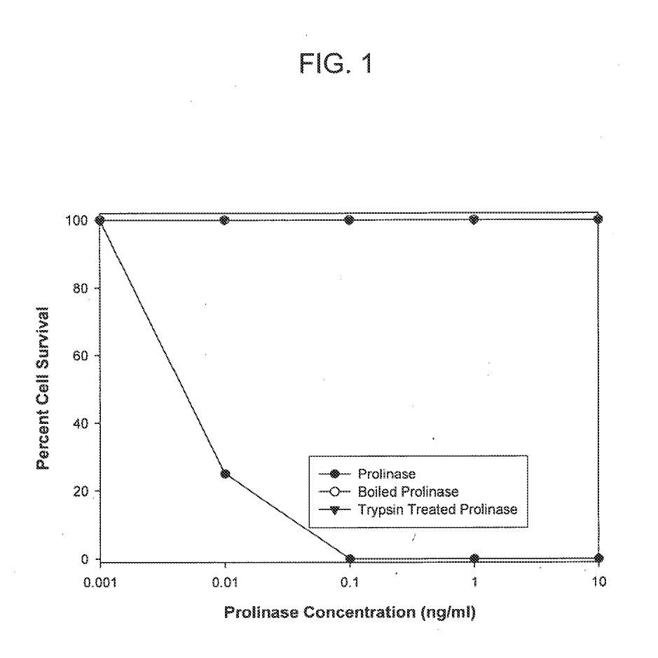

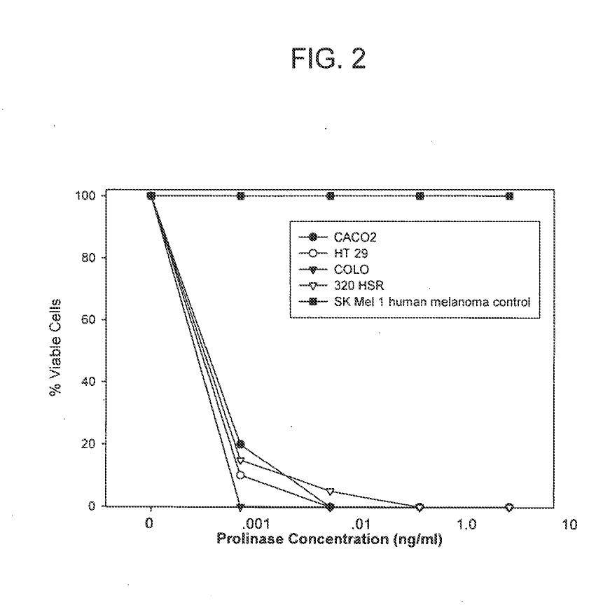

Cells were grown overnight in 96 well plates in growth medium containing both essential and nonessential amino acids. Following the overnight culture, human proline hydroxylase (also referred to as “prolinase” in these Examples; obtained from Sigma Chemical Co., St. Louis, Mo.) was added to wells in quadruplicate, and cells were cultured for an additional three days. Cell viability was determined using methyl thiazolyl tetrazolium (MTT) assays. Incubation with prolinase reduced survival of HT29 cell in a dose-dependent manner (FIG. 1). In particular, FIG. 1 shows no growth in absence of proline and growth in presence of proline Trypsin-treated prolinase and boiled prolinase had no effect on survival (FIG. 1). Prolinase reduced survival of CACO2, HT29, COLO, and 320 HSR colon carcinoma cell lines (FIG. 2). Prolinase did not reduce survival of SK MeI 1 human melanoma cells (FIG. 2).

These data appear to show that prolinase is effective to reduce survival of human carcinoma cells in vitro. Reduced survival correlated with the cells' nutritional requirement for proline and the inability to express pyrroline 5 carboxylate synthase.

### Example 3

### In Vivo Pharmacokinetics of Prolinase

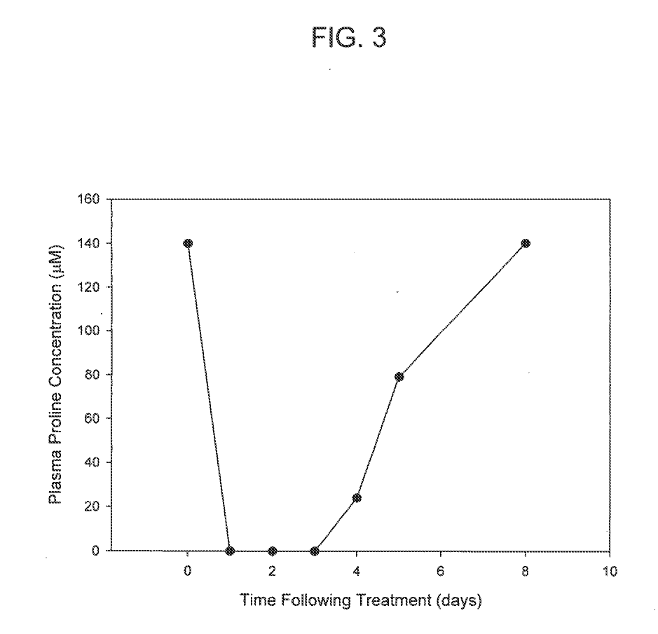

To enhance the circulating half life of prolinase, the enzyme was pegylated in a 20 mM phosphate buffer, pH 8.1, to which a 100 fold excess of succinimidyl PEG 5,000 mw was added. After 30 minutes at room temperature, free PEG was removed by extensive dialysis. Pegylated prolinase was administered intramuscularly (i.m.) to mice, and proline levels in plasma were determined by amino acid analysis. As shown in FIG. 3, administration of pegylated prolinase caused a complete reduction in proline levels between day 0 and day 1. Proline was undetectable in plasma between days 1 and 3. Proline levels returned to pre-administration levels by day 8. The data shown in FIG. 3 are the mean from 5 mice, each injected with 5 IU of pegylated prolinase.

### Example 4

### Tumor Inhibition by Prolinase In Vivo

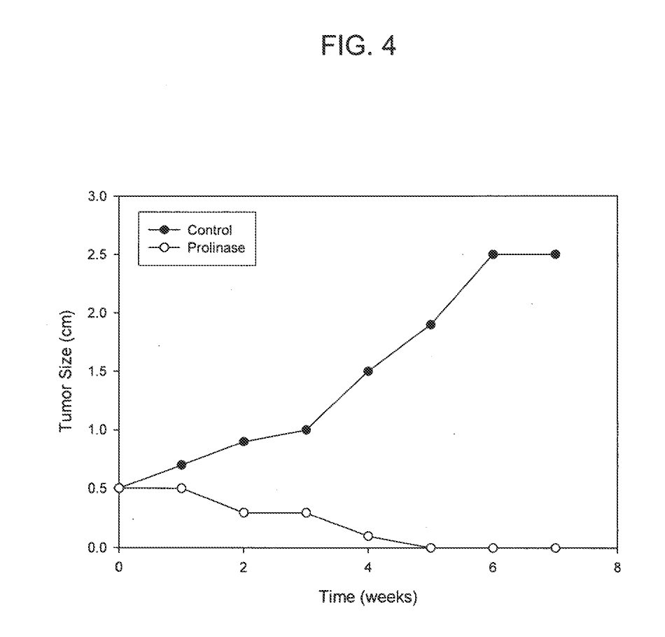

The effects of pegylated prolinase on tumor growth were evaluated in vivo in an animal model. CACO2 human colon carcinomas were implanted subcutaneously into severe combined immunodeficient (SCID) mice. Tumors were allowed to grow to a diameter of 0.5 cm. Pegylated prolinase was administered (5 IU/mouse) once a week for two weeks. Tumor size was measured weekly thereafter. Tumors in mice treated with pegylated prolinase progressively decreased in size (FIG. 4) and were not palpable after five weeks and histologically, only connective tissue was remaining In the control mice, the tumors progressively increased in size, reaching a diameter of 2.5 cm after six weeks (FIG. 4). These data appear to show that pegylated prolinase is effective to reduce tumor size in vivo.

A number of embodiments of the invention have been described. Nevertheless, it will be understood that various modifications may be made without departing from the spirit and scope of the invention. Accordingly, other embodiments are within the scope of the following claims.

What is claimed is:

1. A method of treating a cancer or a cancer symptom in a subject, the method comprising administering to the subject an agent that reduces proline levels in the subject.

2. The method of claim 1, wherein the agent is an enzyme.

3. The method of claim 2, wherein the enzyme is proline hydroxylase.

4. The method of claim 3, wherein the enzyme is modified to increase its circulating half life.

5. The method of claim 4, wherein the enzyme is modified to comprise an Fc region of an immunoglobulin, or a serum albumin.

6. The method of claim 4, wherein the enzyme is linked to one or more polyethylene glycol (PEG) moieties.

7. The method of claim 6, wherein the PEG moiety has a molecular weight of about 5,000 to about 30,000.

8. The method of claim 7, wherein the PEG moiety has a molecular weight of about 5,000.

9. The method of claim 7, wherein the PEG moiety has a molecular weight of 10,000.

10. The method of claim 7, wherein the PEG moiety has a molecular weight of about 20,000.

11. The method of claim 6, wherein the enzyme is linked to three or more PEG moieties.

12. The method of claim 6, wherein the enzyme is linked to one or more PEG moieties by a linking group selected from the group consisting of a succinimide group, an amide group, an imide group, a carbamate group, an ester group, an epoxy group, a carboxyl group, a hydroxyl group, a carbohydrate, a tyrosine group, a cysteine group, a histidine group and a combination thereof.

13. The method of claim 12, wherein the succinimide group is succinimidyl succinate, succinimidyl propionate, succinimidyl carboxymethylate, succinimidyl succinamide, N-hydroxy succinimide or a combination thereof.

14. The method of claim 13, wherein the succinimide group is succinimidyl succinate, succinimidyl propionate or a combination thereof.

15. The method of claim 1, wherein the agent is administered by a route selected from the group consisting of: orally, parenterally, intravenously, intramuscularly, subcutaneously, and intraperitoneally.

16. The method of claim 1, wherein the agent is administered at or near a site of the cancer in the subject.

17. The method of claim 1, wherein the agent is administered in a sustained release formulation.

18. The method of claim 1, wherein the subject is a human.

19. The method of claim 1, wherein the cancer is selected from the group consisting of an ovarian cancer, a colon cancer, a sarcoma, a lymphoma, a myeloma, a breast cancer, prostatic cancer, a skin cancer, an esophageal cancer, a liver cancer, a pancreatic cancer, a uterine cancer, a cervical cancer, a lung cancer, a bladder cancer, and a neural cancer.

20. The method of claim 1, wherein the agent reduces levels of circulating proline by at least 10 μmol/L, 20 μmol/L, 40 μmol/L, 80 μmol/L, 100 μmol/L, or 120 μmol/L.

21. The method of claim 1, wherein the agent is administered in an amount sufficient to reduce growth of cells of the cancer in the subject.

22. The method of claim 1, wherein the agent is administered daily, weekly, every other week, or monthly.

23. A method of treating a cancer or a cancer symptom in a subject, the method comprising reducing dietary proline consumption by the subject.

24. The method of claim 23, further comprising administering a composition comprising an agent that reduces proline levels.

25. The method of claim 24, wherein the agent is selected from the group consisting of an enzyme that reduces proline levels, a compound that increases the expression or activity of an enzyme that catabolizes proline, and an agent that inhibits proline synthesis.

26. The method of claim 24, wherein the agent is proline hydroxylase.

27. A composition for treating a cancer or a cancer symptom, the composition comprising proline hydroxylase linked to one or more PEG moieties.

28. The composition of claim 27, wherein the PEG moiety has a molecular weight of about 5,000 to about 30,000.

29. The composition of claim 27, wherein the one or more PEG moieties has a molecular weight of about 5,000, about 10,000, or about 20,000.

30. The composition of claim 27, wherein the enzyme is linked to three or more PEG moieties.

31. The composition of claim 27, wherein the PEG moiety is linked to proline hydroxylase via a linking group selected from the group consisting of a succinimide group, an amide group, an imide group, a carbamate group, an ester group, an epoxy group, a carboxyl group, a hydroxyl group, a carbohydrate, a tyrosine group, a cysteine group, a histidine group and a combination thereof.

32. The composition of claim 31, wherein the succinimide group is succinimidyl succinate, succinimidyl propionate, succinimidyl carboxymethylate, succinimidyl succinamide, N-hydroxy succinimide or a combination thereof.

33. The composition of claim 32, wherein the succinimide group is succinimidyl succinate, succinimidyl propionate or a combination thereof.

34. The composition of claim 27, further comprising a second agent which is an anti-cancer agent selected from the group consisting of a chemotherapeutic drug and an antibody that induces cytotoxicity in the cancer.

35. A kit for treating a cancer, the kit comprising:
a first agent that reduces proline levels, and
a second agent, wherein the second agent is an anti-cancer agent selected from the group consisting of a chemotherapeutic drug and an antibody that induces cytotoxicity in the cancer.

36. The kit of claim 35, wherein the first agent is proline hydroxylase, an antisense nucleic acid, or a proline analog.
